(19) United States
(12) Patent Application Publication
(10) Pub. No.: US 2023/0033693 A1
(43) Pub. Date: Feb. 2, 2023

(54) MECHANICAL FINGER FOR GRIPPER

(71) Applicant: UNIVERSITÉ LAVAL, Quebec (CA)

(72) Inventors: Vincent BABIN, Quebec (CA); Clément GOSSELIN, Quebec (CA); Thierry LALIBERTE, Quebec (CA)

(21) Appl. No.: 17/789,650

(22) PCT Filed: Jan. 22, 2021
(86) PCT No.: PCT/CA2021/050067

§ 371 Date: Jun. 28, 2022

Related U.S. Application Data
(60) Provisional application No. 62/964,851, filed on Jan. 23, 2020.

(51) Int. Cl. A61F 2/58; B25J 15/00

(52) U.S. Cl. CPC A61F 2/583; B25J 15/0009

(57) ABSTRACT

A mechanical finger has a base. A proximal phalanx and a distal phalanx is provided with a first epicyclic joint between the base and proximal phalanx, and a second epicyclic joint between the proximal phalanx and the distal phalanx. A distal four-bar mechanism includes the proximal phalanx, the distal four-bar mechanism coupled to the distal phalanx. A nail is optionally provided and has a joint mechanism movably connecting the nail to the distal most one of the phalanges between a stowed position in which a grasping tip of the nail is concealed in the distal most one of the phalanges, and a deployed configuration in which the grasping tip projects out of the distal most one of the phalanges.

### CROSS-REFERENCE TO RELATED APPLICATION

The present application claims the priority of U.S. Patent Application No. 62/964,851, filed on Jan. 23, 2020, the entire contents of which are incorporated herein by reference.

### TECHNICAL FIELD

The application relates to mechanical fingers used in robotic applications, such as in grippers and/or as end effectors of robotic arms.

### BACKGROUND

Gripper mechanisms featuring mechanical fingers are nowadays employed in numerous applications. For example, gripper mechanisms are used as end effectors of robotic arms or of robots, in numerous applications, such as industrial robots, assistive robots, automated processes. However, gripper mechanisms may have difficulty grasping a wide diversity of objects. For example, gripper mechanisms may have difficulties in pick up objects laid flat on rigid surfaces when these objects cannot be grasped directly from above. This is particularly true for large flat objects such as books for example. Few solutions exist for the collection of objects of this type.

### SUMMARY

In a first aspect, there is a mechanical finger comprising: a base having a base gear, at least two phalanges, with a first phalanx having a first proximal phalanx gear at a proximal end and a first distal phalanx gear at the distal end, the first proximal phalanx gear operationally coupled to the base gear for the first phalanx to rotate relative to the base gear, and at least a second phalanx having a second proximal phalanx gear at a proximal end, the second proximal phalanx gear operationally coupled to the first distal phalanx gear for the second phalanx to rotate relative to the first phalanx, and a transmission linkage assembly operatively connected to the base and to the at least two phalanges, the transmission linkage assembly including a first carrier between the base gear and the first proximal phalanx gear, and a second carrier between the first distal phalanx gear and the second proximal phalanx gear.

Further in accordance with the first aspect, for instance, the first carrier is part of a first coupling link between the base and the first phalanx, rotational axes of the first coupling link being coincident with a center of the base gear and with a center of the first proximal phalanx gear.

Still further in accordance with the first aspect, for instance, the second carrier is defined by a second coupling link between the first phalanx and the second phalanx, rotational axes of the second coupling link being coincident with a center of the first distal phalanx gear and with a center of the second proximal phalanx gear.

Still further in accordance with the first aspect, for instance, the transmission linkage assembly has at least one four-bar mechanism.

Still further in accordance with the first aspect, for instance, the four-bar mechanism includes the first coupling link, the second coupling link, the first phalanx, and a transmission link.

Still further in accordance with the first aspect, for instance, the first coupling link has rotational joints with the base, the first phalanx and the transmission link, the rotational joints being in a triangular arrangement.

Still further in accordance with the first aspect, for instance, the second coupling link has rotational joints with the first phalanx, the second phalanx and the transmission link, the rotational joints being in a triangular arrangement.

Still further in accordance with the first aspect, for instance, a force sensor is operatively connected to the transmission link.

Still further in accordance with the first aspect, for instance, an underactuation mechanism is configured to interface an actuator to the mechanical finger, whereby the mechanical finger has a passive state of actuation in which a distal most one of the at least two phalanges has a constant orientation relative to the base absent a contact of the phalanges with an object through rotation of the first phalanx relative to the base, and a grasping state of actuation in which a contact of at least one of the phalanges other than the distal most one of the phalanges with an object causes a variation of the orientation of the distal most one of the phalanges relative to the base through rotation of the first phalanx relative to the base.

Still further in accordance with the first aspect, for instance, the underactuation mechanism is a four-bar underactuation mechanism.

Still further in accordance with the first aspect, for instance, the four-bar underactuation mechanism includes the first coupling link.

Still further in accordance with the first aspect, for instance, the four-bar underactuation mechanism is connected to the transmission linkage assembly via a first link shared by the four-bar underactuation mechanism and the transmission linkage assembly.

Still further in accordance with the first aspect, for instance, the first link has rotational joints with the first phalanx, the four-bar underactuation mechanism and the transmission linkage assembly, the rotational joints being in a triangular arrangement.

Still further in accordance with the first aspect, for instance, the transmission linkage assembly has at least one four-bar mechanism.

Still further in accordance with the first aspect, for instance, the four-bar mechanism includes the first link, the second coupling link, the first phalanx, and a transmission link.

Still further in accordance with the first aspect, for instance, the second coupling link has rotational joints with the first phalanx, the second phalanx and the transmission link, the rotational joints being in a triangular arrangement.

Still further in accordance with the first aspect, for instance, a force sensor is operatively connected to the transmission link.

Still further in accordance with the first aspect, for instance, the four-bar underactuation mechanism has a constant shape through rotation of the first phalanx relative to the base in the passive state of actuation.

Still further in accordance with the first aspect, for instance, at least one biasing device and/or a stop in the four-bar underactuation mechanism preserve the constant shape.

Still further in accordance with the first aspect, for instance, the four-bar underactuation mechanism deforms through rotation of the first phalanx relative to the base to cause a rotation of the distal most one of the phalanges in the grasping state of actuation.

Still further in accordance with the first aspect, for instance, at least one biasing device and/or a stop in the four-bar underactuation mechanism return the four-bar underactuation mechanism to a given shape when the object is released.

Still further in accordance with the first aspect, for instance, at least one nail has a joint mechanism movably connecting the nail to the distal most one of the phalanges between a stowed position in which a grasping tip of the nail is concealed in the distal most one of the phalanges, and a deployed configuration in which the grasping tip projects out of the distal most one of the phalanges, and a biasing device for returning the nail to the stowed configuration, wherein the nail is moved to the deployed configuration by rotation of the distal most one of the phalanges relative to a remainder of the mechanical finger as induced by contact of an object by the distal most one of the phalanges.

Still further in accordance with the first aspect, for instance, the mechanical finger has two of the phalanges, the distal most one of the phalanges being the second phalanx.

In accordance with a second aspect, there is provided a mechanical finger comprising: a base, at least a proximal phalanx and a distal phalanx with a first epicyclic joint between the base and proximal phalanx, and a second epicyclic joint between the proximal phalanx and the distal phalanx; and a distal four-bar mechanism including the proximal phalanx, the distal four-bar mechanism coupled to the distal phalanx.

Still further in accordance with the second aspect, for instance, the first epicyclic joint includes a first coupling link between the base and the proximal phalanx.

Still further in accordance with the second aspect, for instance, the second epicyclic joint includes a second coupling link between the proximal phalanx and the distal phalanx.

Still further in accordance with the second aspect, for instance, the distal four-bar mechanism includes the first coupling link, the second coupling link, the proximal phalanx, and a transmission link.

Still further in accordance with the second aspect, for instance, the first coupling link has rotational joints with the base, the proximal phalanx and the transmission link, the rotational joints being in a triangular arrangement.

Still further in accordance with the second aspect, for instance, the second coupling link has rotational joints with the proximal phalanx, the distal phalanx and the transmission link, the rotational joints being in a triangular arrangement.

Still further in accordance with the second aspect, for instance, a force sensor is operatively connected to the transmission link.

Still further in accordance with the second aspect, for instance, an underactuation mechanism is configured to interface an actuator to the mechanical finger.

Still further in accordance with the second aspect, for instance, the underactuation mechanism is a four-bar underactuation mechanism.

Still further in accordance with the second aspect, for instance, the four-bar underactuation mechanism includes the first coupling link.

Still further in accordance with the second aspect, for instance, the four-bar underactuation mechanism is connected to the distal four-bar mechanism via a first link shared by the four-bar underactuation mechanism and the distal four-bar mechanism.

Still further in accordance with the second aspect, for instance, the first link has rotational joints with the proximal phalanx, the four-bar underactuation mechanism and the distal four-bar mechanism, the rotational joints being in a triangular arrangement.

Still further in accordance with the second aspect, for instance, the distal four-bar mechanism includes the first coupling link, the second coupling link, the proximal phalanx, and a transmission link.

Still further in accordance with the second aspect, for instance, the second coupling link has rotational joints with the proximal phalanx, the distal phalanx and the transmission link, the rotational joints being in a triangular arrangement.

Still further in accordance with the second aspect, for instance, a force sensor is operatively connected to the transmission link.

Still further in accordance with the second aspect, for instance, the four-bar underactuation mechanism has a constant shape through rotation of the proximal phalanx relative to the base in the passive state of actuation.

Still further in accordance with the second aspect, for instance, at least one biasing device and/or a stop in the four-bar underactuation mechanism preserves the constant shape.

Still further in accordance with the second aspect, for instance, the four-bar underactuation mechanism deforms through rotation of the proximal phalanx relative to the base to cause a rotation of the distal most one of the phalanges in the grasping state of actuation.

Still further in accordance with the second aspect, for instance, at least one biasing device and/or a stop is in the four-bar underactuation mechanism to return the four-bar underactuation mechanism to a given shape when the object is released.

Still further in accordance with the second aspect, for instance, at least one nail has a joint mechanism movably connecting the nail to the distal most one of the phalanges between a stowed position in which a grasping tip of the nail is concealed in the distal most one of the phalanges, and a deployed configuration in which the grasping tip projects out of the distal most one of the phalanges, and a biasing device for returning the nail to the stowed configuration, wherein the nail is moved to the deployed configuration by rotation of the distal most one of the phalanges relative to a remainder of the mechanical finger as induced by contact of an object by the distal most one of the phalanges.

In accordance with a third aspect, there is provided a nail system for a distal phalanx of a mechanical finger, comprising at least one nail having a joint mechanism movably connecting the nail to the distal phalanx between a stowed position in which a grasping tip of the nail is concealed in the distal phalanx, and a deployed configuration in which the grasping tip projects out of the distal phalanx, and a biasing member for returning the nail to the stowed configuration, wherein the nail is moved to the deployed configuration by rotation of the distal phalanx relative to a remainder of the mechanical finger as induced by contact of an object by the distal phalanx.

Still further in accordance with the third aspect, for instance, the distal phalanx includes a pair of plates, with the nail slidingly positioned between plates.

Still further in accordance with the third aspect, for instance, the distal phalanx is connected to a gear configured to form an epicyclic joint with a phalanx adjacent to the distal phalanx.

Still further in accordance with the third aspect, for instance, the distal phalanx is rotatably connected to the gear, and is biased to a given orientation relative to the gear by the biasing member.

Still further in accordance with the third aspect, for instance, the nail system includes a member received in at least one slot of the nail, the member engaged with the gear such that a rotation of the distal phalanx relative to the gear imparts a movement of the nail to the deployed configuration via a cooperation of the member and the slot.

Still further in accordance with the third aspect, for instance, a cam and follower surface assembly is between the nail and the gear, the cam being in operative contact with the follower surface, whereby a rotation of the distal phalanx relative to the gear imparts a movement of the nail to the deployed configuration via a cooperation of the cam and follower surface.

Still further in accordance with the third aspect, for instance, the follower surface is peripheral part of the nail.

### DESCRIPTION OF THE DRAWINGS

Reference is now made to the accompanying figures in which:

FIG. 1 is a side elevation view of a gripper finger in accordance with an aspect of the present disclosure;

FIG. 2 is a side elevation view of a gripper finger in accordance with another aspect of the present disclosure, with an underactuation mechanism;

FIG. 3 is a schematic view of the gripper finger of FIG. 2, with a translational degree of actuation;

FIG. 4 is a grasping sequence view of the gripper finger of FIG. 2, without activation of an underactuation mechanism;

FIG. 5 is a grasping sequence view of the gripper finger of FIG. 2, with activation of the underactuation mechanism;

FIG. 6 is an assembly view of an embodiment of the gripper finger of FIG. 2;

FIG. 7 is a perspective view of a distal phalanx of the gripper fingers of FIG. 1 and/or FIG. 2, showing a nail system thereon;

FIG. 8 is a sequence view of the distal phalanx of FIG. 7, showing a deployment of a nail of the nail system;

FIG. 8A is a sequence view of the distal phalanx of FIG. 7, showing a deployment of a variant of a nail of the nail system;

FIG. 9 is an assembly view of the distal phalanx and nail system of FIG. 8;

FIG. 10 is a sequence view of a gripper mechanism having the distal phalanx and nail system of FIG. 7;

FIG. 11 is a perspective view of a gripper mechanism having a pair of the gripper fingers of FIG. 1;

FIG. 12 is a perspective view of a gripper mechanism having a pair of gripper fingers of FIG. 1; and

FIG. 13 has schematic graphs showing a rationale for a location of a sensor in the gripper finger of FIG. 1.

### DETAILED DESCRIPTION

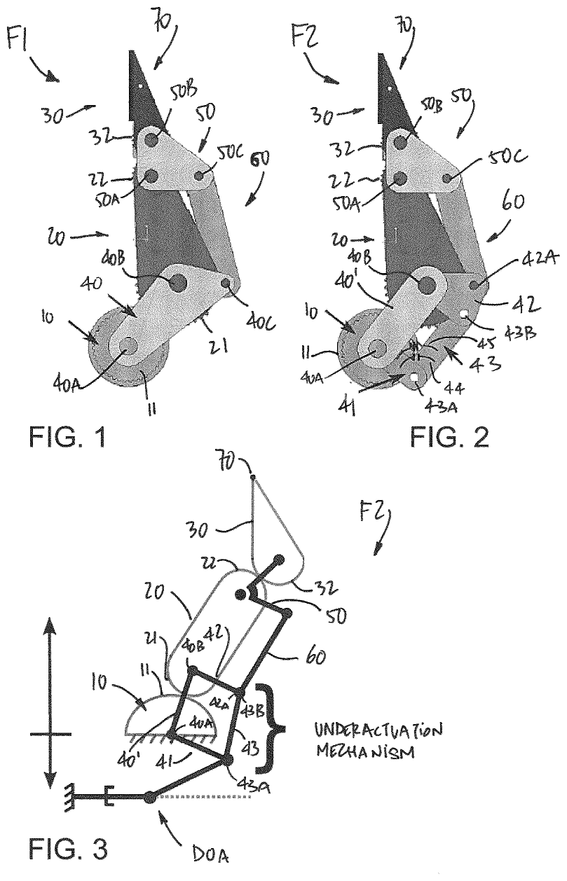

Referring to the drawings and more particularly to FIGS. 1 and 2, variants of a gripper finger are shown at F1 and F2, respectively. The fingers F1 and F2 are referred to as gripper fingers as they may be part of a gripper, also known as a gripping system, a robotic hand, a robot, an end effector or member of a robotic arm, etc. Reference is made herein to a gripper for simplicity, but it is understood that the fingers could be used in other types of mechanisms. Moreover, the expressions “proximal” and “distal” are used herein, with proximal being closer to base of the fingers F1 and F2, are distal being closer to a tip of the fingers F1 and F2.

As a possibility, the fingers F1 and F2 are used in pairs, e.g., one finger F1 and one finger F2 used concurrently as part of a gripper, though more of the fingers could be present, or fingers F1 only or F2 only. Examples thereof are provided hereinafter, but to simply the description, FIGS. 1 to 5 will detail a single finger at a time. The gripper finger F1 has a base 10, a proximal phalanx 20, a distal phalanx 30, a first coupling link 40, a second coupling link 50, a transmission link 60, and optionally a nail system 70. The gripper finger F1 could have an additional or additional phalanges, distally connected to the distal phalanx 30, with additional coupling links. The phalanges 20, 30 and the links 40, 50 and 60 are all links in accordance with a mechanical definition thereof, i.e., links are the rigid members connected by joints. The joints (also called axes) are the movable components of a robot that cause relative motion between adjacent links. Links space apart joints. In embodiments of the fingers F1 and F2, the interconnection between base 10 and the phalanx 20 includes epicyclic joints, such as an epicyclic gear train. Likewise. In embodiments of the fingers F1 and F2, the interconnection between the phalanx 20 and the phalanx 30 may also include an epicyclic joint. An epicyclic joint may be defined as a joint in which one component, such as the phalanx 20, rotates around a center of another component, such as the base 10, with a carrier interconnecting the two components at their respective centers, and rotating while carrying one component (e.g., the phalanx 20). An epicyclic gear train may be known as a planetary gearset, and may herein consists of two gears mounted so that the center of one gear revolves around the center of the other. A carrier connects the centers of the two gears, the carrier rotating to carry one gear. The expressions “epicyclic motion” and “epicycloidal motion” pertain to the motion of one component (e.g., gear) relative to the other (e.g., other gear in a two-gear epicyclic gear train). Other types of mechanisms may form epicyclic joints, enabling an epicycloidal motion may be used where appropriate, such mechanisms constraining two links to undergo an epicycloidal motion with respect to one another. For example, an epicyclic joint may include a pair of pulleys and a belt, cogged wheels (a.k.a., cogs or chainring and a chain).

The phalanges 20 and 30 (and others if present) define the body parts of the finger F1, as they are the contact interfaces of the finger F1 with objects the finger F1 will grasp, for instance in collaboration with other fingers, along with the nail system 70 (if present). The links 40, 50 and 60 form a transmission linkage assembly, and interface the phalanges 20 and 30 with an actuator, to cause movement of the phalanges in a manner described herein. The transmission linkage assembly may include fewer or more than the links 40, 50 and 60, and other components such as the joints between links, stoppers, biasing devices, etc, as described herein.

In the illustrated embodiment, the epicyclic joints in the fingers F1 and F2 are epicyclic gear trains, though other types of epicyclic joints may be used as described above. The base 10 is shown as being defined by a base gear 11. The base gear 11 may be referred to as a base pulley, base wheel, base cog, etc. As in FIG. 1, the base gear 11 may be a full spur gear (i.e., extending 360 degrees) or may be a section of a full gear, such as a semicircular gear, etc. The proximal phalanx 20 has a proximal phalanx gear 21 at its proximal end. The proximal phalanx gear 21 is shown as being a spur gear extending around less than 360 degrees, and alternatives are contemplated, such as complementary helical gears for the base gear 11 and the proximal phalanx gear 21. The proximal phalanx gear 21 is operatively coupled to the base gear 11, i.e., they are intermeshed by their complementary gear parameters, such as number of teeth, pitch, tooth thickness, etc. Accordingly, the proximal phalanx gear 21 may move along the base gear 11 by rotating along its rotational axis. The proximal phalanx gear 21 and the base gear 11 are in an epicyclic relation, with the proximal phalanx gear 21 revolving around the center of the base gear 11. The first coupling link 40 acts as epicicylic carrier between the base gear 11 and the proximal phalanx gear 21.

Still referring to FIG. 1, the proximal phalanx 20 has a distal phalanx gear 22 at its distal end. The distal phalanx gear 22 is operatively coupled to a proximal phalanx gear 32 at a proximal end of the distal phalanx 30. Hence, the proximal phalanx 20 and the distal phalanx 30 are intermeshed with the complementary gear parameters of the distal phalanx gear 22 and proximal phalanx gear 32. The proximal phalanx gear 32 may move along the distal phalanx gear 22 by rotating along its rotational axis, in an epicyclic relation, with the proximal phalanx gear 32 revolving around the center of the distal phalanx gear 22. The second coupling link 50 acts as epicyclic carrier between the distal phalanx gear 22 and the proximal phalanx gear 32. The gears 22 and 32 extend on less than 360 degrees, and may be spur gears, helical gears, etc.

The first coupling link 40 is pivotally connected at joint 40A to the base 10, and at joint 40B to the proximal phalanx 20. In a variant, rotational axes of the first coupling link 40 are coincident with a center of the base gear 11 and with a center of the proximal phalanx gear 21. Furthermore, the first coupling link 40 is pivotally connected to the transmission link 60 at joint 40C. Joints 40A, 40B and 40C may be pivot joints. In an embodiment, the rotational axis of the joints 40A, 40B and 40C are parallel to one another. The first coupling link 40 may be connected to an actuator in any appropriate way to cause a movement of the phalanges 20 and/or 30. The actuator may for instance provide a single degree of actuation (DOA) to the first coupling link 40, which single DOA may suffice in performing grasping of objects with the finger F1, and another finger.

The second coupling link 50 is pivotally connected at joint 50A to the proximal phalanx 20, and at joint 50B to the distal phalanx 30. In a variant, rotational axes of the second coupling link 50 are coincident with a center of the distal phalanx gear 22 and with a center of the proximal phalanx gear 32. Furthermore, the second coupling link 50 is pivotally connected to the transmission link 60 at joint 50C. Joints 50A, 50B and 50C may be pivot joints (i.e., revolute joints). In an embodiment, the rotational axes of the joints 50A, 50B and 50C are parallel to one another. The transmission link 60 is connected at its opposed ends to the first coupling link 40 and to the second coupling link 50, via joints 40C and 50C respectively. As observed, the transmission link 60 is part of a four-bar mechanism, including joints 40B, 40C, 50A and 50C, which four-bar mechanism may be a parallelogram to maintain a constant orientation of the distant phalanx 30 as described below. The transmission link 60 transmits forces to the second coupling link 50.

The nail system 70 is located on the distalmost phalanx, in finger F1 the distal phalanx 30, and may selectively be deployed to assist in grasping some types of objects, as detailed below. For instance, the distal phalanx 30 may have a high friction surface (e.g., rubber or like polymer) so as to provide sufficient grasping adherence to support some objects. In contrast, the nail system 70 may have a hard and relatively low friction surface, such as a metal or high density polymer.

Referring now to FIG. 2, the finger F2 is described. The finger F2 has numerous components in common with the finger F1, whereby like reference numerals will indicate like elements. One difference may be with the presence of an underactuation mechanism including a four-bar mechanism (e.g., referred to as a four-bar underactuation mechanism), between the base 10 and the proximal phalanx 20. In the illustrated embodiment, the epicyclic joint in the finger F2 are epicyclic gear trains, though other types of epicyclic joints may be used as described above.

In the finger F2 of FIG. 2, the base 10 is shown as being defined by a base gear 11, a.k.a., a base pulley, base wheel, base cog, etc. As in FIG. 1, the base gear 11 may be a full spur gear (i.e., extending 360 degrees) or may be a section of a full gear, such as a semicircular gear, etc. The proximal phalanx 20 has a proximal phalanx gear 21 at its proximal end. The proximal phalanx gear 21 is shown as being a spur gear extending around less than 360 degrees, and alternatives are contemplated, such as complementary helical gears for the base gear 11 and the proximal phalanx gear 21. The proximal phalanx gear 21 is operatively coupled to the base gear 11, i.e., they are intermeshed by their complementary gear parameters, such as number of teeth, pitch, tooth thickness, etc. Accordingly, the proximal phalanx gear 21 may move along the base gear 11 by rotating along its rotational axis. The proximal phalanx gear 21 and the base gear 11 are in an epicyclic relation, with the proximal phalanx gear 21 revolving around the center of the base gear 11. The first coupling link 40′ acts as epicyclic carrier between the base gear 11 and the proximal phalanx gear 21.

Still referring to FIG. 2, the proximal phalanx 20 has a distal phalanx gear 22 at its distal end. The distal phalanx gear 22 is operatively coupled to a proximal phalanx gear 32 at a proximal end of the distal phalanx 30. Hence, the proximal phalanx 20 and the distal phalanx 30 are intermeshed with the complementary gear parameters of the distal phalanx gear 22 and proximal phalanx gear 32. The proximal phalanx gear 32 may move along the distal phalanx gear 22 by rotating along its rotational axis, in an epicyclic relation, with the proximal phalanx gear 32 revolving around the center of the distal phalanx gear 22. The second coupling link 50 acts as epicyclic carrier between the distal phalanx gear 22 and the proximal phalanx gear 32. The gears 22 and 32 extend on less than 360 degrees, and may be spur gears, helical gears, etc.

The first coupling link 40′ is part of the underactuation mechanism. The first coupling link 40′ is pivotally connected at joint 40A to the base 10, and at joint 40B to the proximal phalanx 20. In a variant, rotational axes of the first coupling link 40 are coincident with a center of the base gear 11 and with a center of the proximal phalanx gear 21. The underactuation mechanism may further include link 41, link 42 and link 43, concurrently arranged with the first coupling link 40′ in a four-bar mechanism. Link 41 shares a rotational axis with the first coupling link 40′ at joint 40A (i.e., rotational axes are coincident), while link 42 shares a rotational axis with the first coupling link 40′ at joint 40B (i.e., rotational axes are coincident). Link 43 is pivotally connected at its opposed ends to the link 41 and 42, respectively by joints 43A and 43B. Furthermore, the link 42 is pivotally connected to the transmission link 60 at joint 42A. Link 42 may therefore have three rotational axes, in a triangular pattern, in a variant, with other embodiments considered. Joints 40A, 40B, 42A, 43A and 43B may be revolute joints, also known as pivot joints. In an embodiment, the rotational axes of the joints 40A, 40B, 42A, 43A and 43B are all parallel to one another. Any part of the underactuation mechanism may be connected to an actuator in any appropriate way to cause a movement of the phalanges 20 and/or 30. The actuator may for instance provide a single degree of actuation (DOA) to the underactuation mechanism, which single DOA may suffice in performing grasping of objects with the finger F2, and another finger. FIG. 3 shows the DOA as being for example a translational DOA, with a linkage assembly connected to the joint 43A to transmit the DOA. Other arrangements are possible, such as by having the base gear 11 connected to a rotational DOA (e.g., directly or via a transmission), with the base gear 11 rigidly connected to the link 41.

The underactuation mechanism may further include a stop 44 and/or a biasing device 45. The stop 44 and the biasing device 45 are shown schematically in FIG. 2, but may take various forms. In an embodiment, the stop 44 is an abutment on one of the links 40-43, and the biasing device 45 is a spring exerting a biasing force to pull adjacent links toward one another such that the stop 44 prevents further movement. For instance, as shown, the stop 44 is on one or both of the links 41 and 43, and the biasing device 45 is also connected at its opposed ends to the links 41 and 43 to force them against one another at the stop 44. The stop 44 and the biasing device 45 could be elsewhere in the underactuation mechanism 40, and could take other shapes. The underactuation mechanism 40 may rely on friction at its joints to maintain its shape, as an option.

The second coupling link 50 is pivotally connected at joint 50A to the proximal phalanx 20, and at joint 50B to the distal phalanx 30. In a variant, rotational axes of the second coupling link 50 are coincident with a center of the distal phalanx gear 22 and with a center of the proximal phalanx gear 32. Furthermore, the second coupling link 50 is pivotally connected to the transmission link 60 at joint 50C. Joints 50A, 50B and 50C may be pivot joints. In an embodiment, the rotational axis of the joints 50A, 50B and 50C are parallel to one another. The transmission link 60 is connected at its opposed ends to the link 42 and to the second coupling link 50, via joints 42A and 50C, respectively. The transmission link 60 transmits forces to the second coupling link 50. As observed, the transmission link 60 is part of a four-bar parallelogram, including joints 40B, 42A, 50A and 50C.

The nail system 70 may also be present in the distalmost phalanx, in finger F2 the distal phalanx 30, and may selectively be deployed to assist in grasping some types of objects, as detailed below. Finger F2 may also be without the nail system 70.

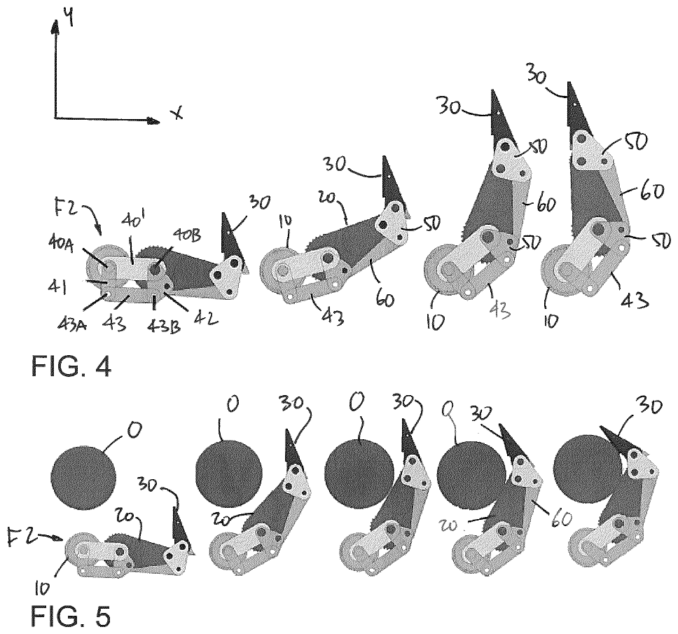

Referring to FIGS. 4 and 5, two sequences of figures are shown to illustrate a movement of the finger F2 without activation of the underactuation mechanism (FIG. 4), and with activation of the underactuation mechanism (FIG. 5). Finger F1 may exhibit a similar behavior as in FIGS. 4 and 5, though without the presence of the underactuation mechanism, and therefore with other means of actuation.

In FIG. 4, when no contact is present on the proximal phalanx 20, the four-bar linkage of the underactuation mechanism remains in a constant configuration, i.e., it does not deform due to the concurrent action of the stop 44 and biasing device 45. The underactuation mechanism is sized to cause a deformation of the other four-bar mechanism, i.e., that includes joints 40B, 42A, 50A and 50C. As a result, the distal phalanx 30 maintains a constant orientation while moving, as observed in FIG. 4. Constant orientation for a position variation may be viewed as per the plane of the distal phalanx 30 having the same angle relative to a referential system X-Y, the plane being parallel to Y as an example—the plane could maintain a constant orientation at another angle. Stated differently, the plane of the distal phalanx 30 is parallel in all images of FIG. 4.

By contrast, as shown in the images of FIG. 5, an enveloping grasp is obtained when a contact is present on the proximal phalanx 20. As the proximal phalanx 20 contacts object O, the proximal phalanx 20 is blocked from rotating further. This causes the input movement to be transmitted directly to the distal phalanx 30, via deformation of the underactuation mechanism (i.e., away from the constant configuration), making the distal phalanx 30 close inward towards the object O. The deformation is against the force of the biasing device 45. The underactuation mechanism therefore allows the underactuation of the finger F2, as the movements of both FIG. 4 and FIG. 5 are possible via a single DOA, i.e., two distinct movements of the fingers F2 for a same DOA. The finger F2 may thus be described as a finger with epicycloidal mechanisms, featuring a four-bar linkage located at the base 10 of the finger F2, providing the required extra freedom while not interfering with the F2 that maintains the distal phalanx 30 in a constant orientation when no contact occurs with the environment. When a pair of fingers F2 are used face to face, the distal phalanges 30 of the two fingers F2 may remain in constant orientation relative to one another, until an object contacts one of the proximal phalanges 20. One arrangement has the distal phalanges 30 parallel to one another, but other arrangements are possible as well.

According to an embodiment, the finger F2 may be regarded as two interconnected four-bar mechanisms, with a proximal four-bar mechanism forming part of a proximal phalanx, and with a distal phalanx connected to the distal four-bar mechanism, and with epicyclic interconnections between the base and proximal phalanx, and between the proximal phalanx and the distal phalanx. In another embodiment of the finger F2, there are three or more phalanges, with epicyclic interconnections between adjacent phalanges. In such a scenario, there may be as many four-bar mechanisms as there are phalanges, with the same arrangement as described above, with a link such as link 42 interconnecting adjacent four-bar mechanisms.

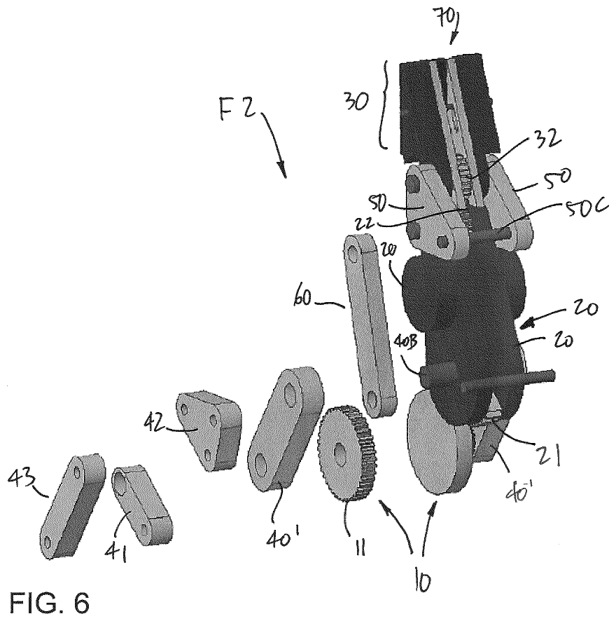

Referring to FIG. 6, there is shown an assembly view for some of the links of the finger F2. This is provided as one contemplated embodiment among others. As shown, the link 42 may be embodied by a pair of plates with a gap between, sharing pins acting as the joints, such as pin/joint 42A. The second coupling link 50 may also be defined by a pair of plates 50. The proximal phalanx 20 is shown as including a pair of plates 20 forming a gap between them, with the proximal phalanx gear 21 and the distal phalanx gear 22 rigidly received in the gap, so as not to rotate. In another embodiment, the proximal phalanx 20 has a monoblock construction. The link 40′ may also be a pair of links, as shown.

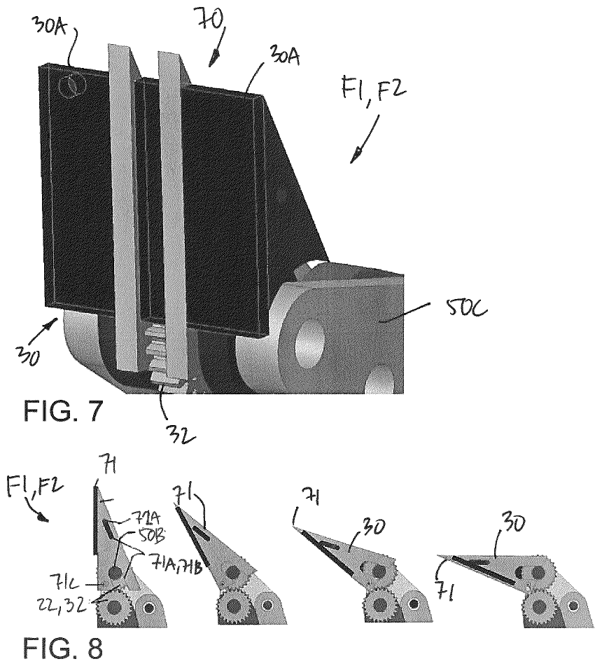

Referring now to FIGS. 7 to 10, the nail system 70 is shown in greater detail, relative to the distal phalanx 30. The nail system 70 may be integrated into the distal phalanx 30 of the finger F1, or of the finger F2. As shown in FIG. 7, the distal phalanx 30 may have a generally planar grasping surface, though the surface may have other surface geometries and peripheries. The distal phalanx 30 may be defined by a pair of plates, both shown as 30, mounted to a pin forming joint 50B. The plates 30 may be spaced apart for the proximal phalanx gear 32 to be located between them, free to rotate relative to the plates 30. The proximal phalanx gear 32 is meshed with the gear 22 and hence is rigidly linked to the movement of the proximal phalanx 20, in an epicyclic manner with the second coupling link 50 acting as carrier. In terms of rotation, distal phalanx 30 and the nail system 70 keep the same orientation with respect to one another but rotate with respect to the proximal phalanx gear 32. The nail(s) 71 of the nail system 70 slides with respect to the distal phalanx 30 along the slots 71A and 71B thus always keeping the same orientation with respect to the distal phalanx 30. The surfaces of the distal phalanx 30 may include pads 30A. In an embodiment, the pads 30A are a rubber or like elastomer or polymer, with elastic deformation capability and relative high friction. Other variations are possible, including harder materials, or the absence of pads 30A altogether.

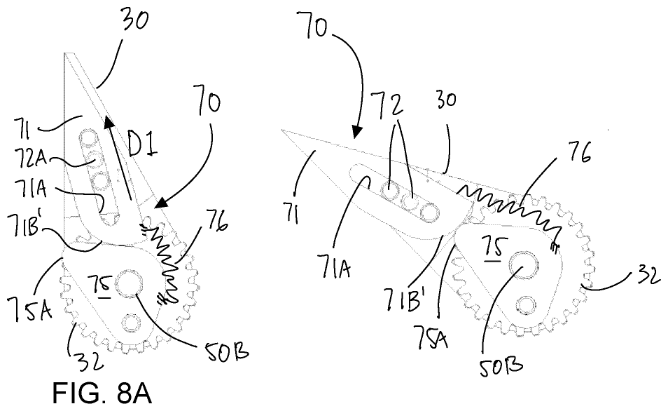

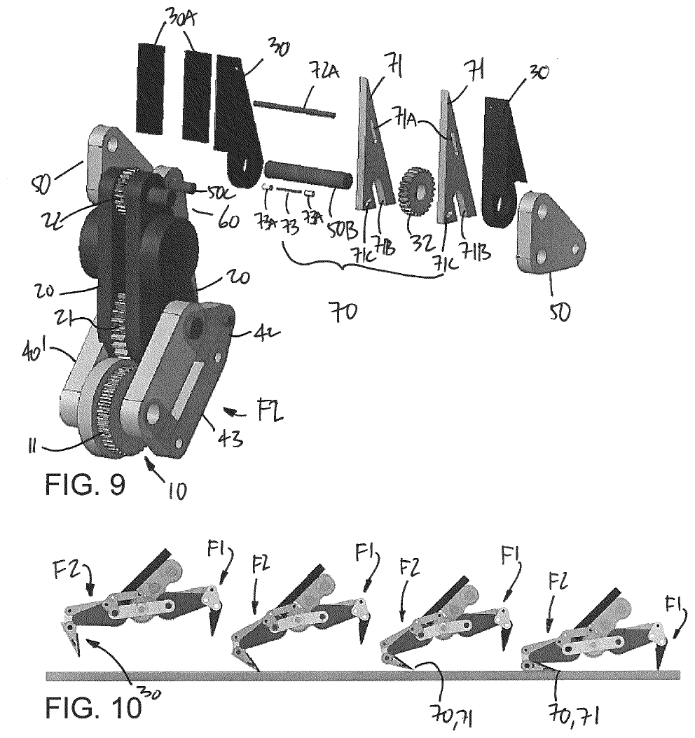

Referring to FIGS. 7 and 9, the nail system 70 has one or more nails 71. Different mechanisms may be used to deploy the nail 70, one of which mechanisms is shown in FIG. 8 while the other is shown in FIG. 8A. The nails 71 may be plates that emulate the distal contour of the plates 30 forming the distal phalanx 30. Other geometries are considered as well. Therefore, when the nail system 70 is in its stowed configuration, as in the left hand size image of FIG. 8 and of FIG. 8A, the nail(s) 71 are concealed in the distal phalanx 30 so as not to project out of the distal phalanx 30. Referring to FIGS. 8 and 9, the nails 71 may have a guide slot 71A in collaboration with a guide pin 72A (or like guide and follower assembly), the guide pin 72A being fixed in position to the distal phalanx 30. Another guide slot 71B may also be in the nails 71, and may for instance use the pin of the joint 50B as guide. One guide slot/guide may suffice, or other guide and sliding surface pairs may be used as alternatives to the ones described above. In the embodiment described in FIGS. 8 and 9, the guide slots 71A and 71B are parallel to one another, and define a trajectory of movement of the nail(s) relative to the distal phalanx 30. The trajectory allows the selective concealing of the nails 71 in the distal phalanx.

Moreover, a driving pin or like member 73 is coupled to a pair of slots 71C in the nails 71. The driving pin 73 is held captive in the slots 71C, for instance by bushings 73A or with enlarged heads (e.g., bolt and nut assembly). The slots 71C are not parallel to the guide slots 71A and 71B. Moreover, the slots 71C are in a non-parallel orientation relative to the direction of movement of the nails 71. The driving pin 73 is positioned between teeth of the proximal phalanx gear 32, or passes through a bore in the proximal phalanx gear 32.

With reference to FIGS. 8 and 10, during the deployment of the nails 71 from the stowed configuration, the parts kept in place during activation are the distal phalanx gear 22, the proximal phalanx gear 32 and the driving pin 73. As mentioned above, the guide pin 72A is rigidly attached to the distal phalanx 30. By contact with a surface, the distal phalanx 30 may be caused to rotate relative to the proximal phalanx 20. For instance, this occurs when the distal phalanx 30 contacts a surface, as in FIG. 10. As the distal phalanx 30 rotates, while the distal phalanx gear 22, the proximal phalanx gear 32 and the driving pin 73 do not rotate as retained by the proximal phalanx gear 32, a camming action occurs by the sliding movement of the driving pin 73 in the slots 71C of the nails 71. This causes a simultaneous movement of the nails 71 sliding along the pin 71A and pin of the joint 50B. Consequently, the nails 71 gradually project out from stowing. The apex of the nails 71 is well suited to slide under objects laid flat on a surface, as the apex of the nails 71 may act as a wedge. Although not shown, a biasing device may be present to bias the nails 71 to the concealed position of the left-hand side. The biasing device may be for example a coil spring between each of the nails 71 and parts of the distal phalanx 30 that do not move with the nails 71, such as a the pin 71A, as one possible example.

Referring to FIG. 8A, another embodiment of the nail system 70 is shown, whereby like reference numerals will identify like elements. In FIG. 8A, the nail 71 may have a guide slot 71A in collaboration with a guide pin(s) 72A (or like guide and follower assembly) on the distal phalanx 30. The nail 71 may therefore slide relative to the guide pin(s) 72A, in the direction shown by D1 for the deployment. The nail 71 may include a follower surface 71B′, the follower surface 71B′ also known as a contact surface, periphery, etc. A cam 75 is secured to the proximal phalanx gear 32 for concurrent rotation. A cam portion 75A of the cam 75 is in sliding contact with the follower surface 71B′ of the nail 70. The cam and following surface assembly may be reversed, with the cam being part of nail, for example. A biasing member 76, such as a coil spring, extends between the nail 71 (or plates 30 of the phalanx 30) and the cam 75 (or the proximal phalanx gear 32). Therefore, without given contacts of the distal phalanx gear 32 with a surface (as in FIG. 10), the biasing member 76 biases the nail 71 to the stowed configuration of the left-hand side image of FIG. 8A. The biasing member 76 may also contribute to preserve a constant orientation between the plates of the distal phalanx 30 and the distal phalanx gear 32. As a surface is contacted as in FIG. 10, the plates 30 of the distal phalanx 30 may rotate relative to the distal phalanx gear 32 (via pin of the joint 50B, FIG. 9), against the force of the biasing member 76. The rotation will entrain the nail 71, but as the nail 71 may translate and is in contact with the cam 75, the nail 71 may slide to its deployed configuration, as in the right-hand side image of FIG. 8A. In an embodiment, a leading edge of the nail 71 remains parallel to the leading edge of the distal phalanx 70. When the given contact with the surface is released, the biasing member 76 returns the distal phalanx 30 and nail 71 to the stowed configuration of the left-hand side image of FIG. 8A.

The nail system 70 provides another degree of freedom (DOF) to the fingers F1 and F2, though a non-actively actuated DOF. It is by contact of a surface, for example, that the nails 71 of the nail system 70 move to their deployed configuration. The other DOF provided by the nail system 70 is compliant with the surface contacted by the fingers F1 or F2. Stated differently, the nail system 70 has one or more nails 71 having a joint mechanism, such as with the slots 71 and pins 50B, 72A, and 73, for example, movably connecting the nail 71 to the distal phalanx 30 between a stowed position in which a grasping tip of the nail 71 is concealed in the distal phalanx 30, and a deployed configuration in which the grasping tip projects out of the distal phalanx 30. A biasing device 76 may be provided to return the nail 71 to the stowed configuration. The nail 71 is moved to the deployed configuration by rotation of the distal phalanx 30 relative to a remainder of the mechanical finger F1 or F2 as induced by contact of an object by the distal phalanx 30.

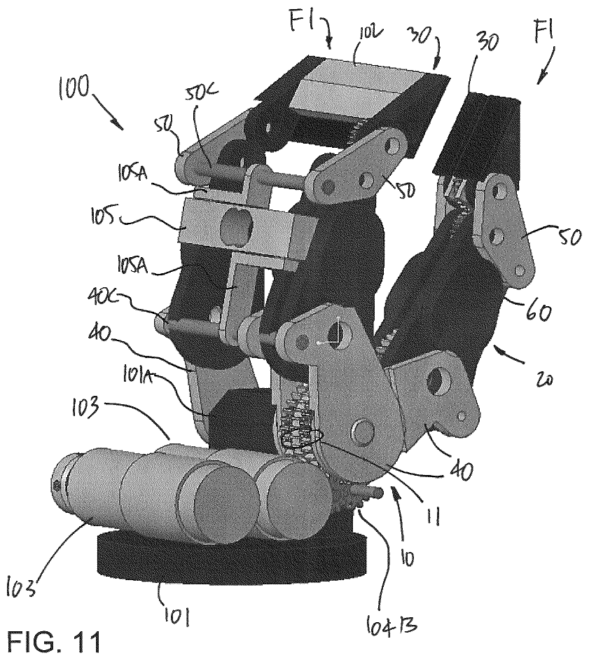

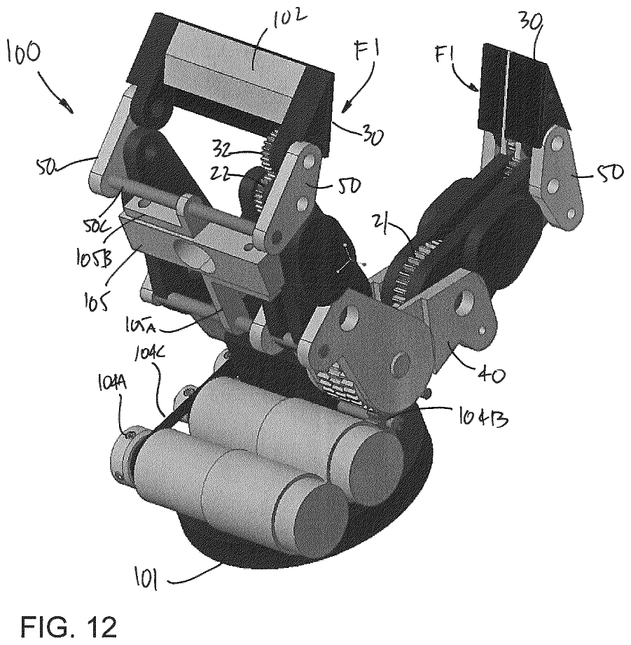

Referring to FIGS. 11 and 12, an exemplary gripper mechanism is shown at 100. The gripper mechanism 100 is illustrated as an example of the integration of a pair of the fingers F1 into a tool. While the description refers to the fingers F1, a similar arrangement may be used with the fingers F2, considering that no additional DOA may be required to operate the fingers F2 in spite of the additional freedom of movement permitted by the presence of the underactuation mechanism. In the gripper mechanism 100, a platform 101 may be used to support both fingers F1 (or F2), face to face. The platform 101 may have a mount 101A, for the connection of the bases 11 of the fingers F1 to the platform 101. For example, in FIGS. 11 and 12, the fingers F1 share a common axis of rotation for the first coupling links 40, on the mount 101A, though the axes of rotation could be spaced apart as well. It is observed that one of the fingers F1 is larger than the other. This may be achieved by having longer pins for the various joints, and spacer blocks, such as the one shown at 102 for the distal phalanx 30.

In an embodiment, there are two DOAs, i.e., one per finger F1 and/or F2. The DOAs may be embodied by motors 103 mounted to the platform 101, and coupled to the fingers F1 and/or F2 by an appropriate transmission, featuring components such as pulleys 104A, gears 104B, belt 104C etc. It is contemplated to have a single DOA, such as a bidirectional motor coupled by transmission to both fingers F1 and/or F2.

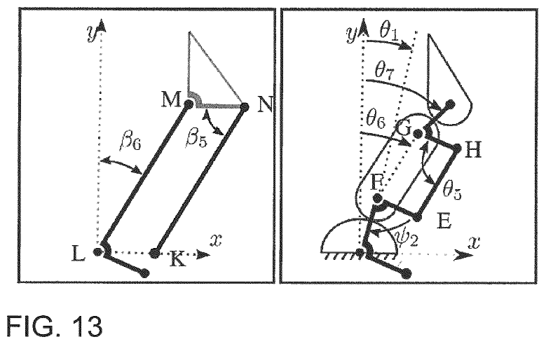

A force sensor 105 is shown in FIGS. 11 and 12, in one possible location, in the transmission link 60 (other locations are also contemplated). Since the gripper mechanism 100 interacts with the environment, sensing devices are needed in order to make the interactions with the environment safe and non-destructive. According to an embodiment, if located strategically in the gripper mechanism 100, a single force component measured together with assumptions on the direction of that force may be sufficient to implement the scooping method and the sensing of the grasping forces. The sensor 105 is mounted to the pins of pivots 40C and 50C, via brackets 105A. In a variant, the assembly of the sensor 105 and its brackets 105A may replace the transmission link 60. Other locations are considered, and in the case of finger F2, the load cell 105 may be mounted to the pins of pivots 42A and 50C, for example. The placement of the sensor 105 as in FIGS. 11 and 12 may be better than in conventional four-bar parallel grippers because the four-bar parallel grasp mechanism of the fingers F1 and F2 is driven at both ends by the epicyclic gears. Referring to FIG. 13, a comparison is made between a four-bar parallel grasp mechanism used in a standard gripper on the left-hand side and the arrangement of finger F1 (as applicable to F2) on the right-hand side.

For the conventional four-bar finger on the left of FIG. 13, it is seen that for each increment of β₆, dβ₆, increments of β₅, dβ₅ will decrease of an equal amount. As a consequence, the wider a gripper with the conventional four-bar finger is opened, the closer the lines MN and KN are brought together. This transmits larger forces through the sensor, but may give rise to interference problems because parts approach each other too much.

The fingers of present disclosure are exemplified by finger F1 on the right-hand side FIG. 13. To compare with the conventional four-bar finger of the left-hand side, the relation between increments deb and des should be considered. The equation relating the two increments of angle is:

$$d θ \text{?} = - \frac{{R}_{1}}{{R}_{1} + \text{?}} d θ \text{?}$$

$$\text{?} \text{indicates text missing or illegible when filed}$$

where R₁ is a ratio of the planetary which in the fingers F1 and F2 is 1, hence the relation is

dθ₃=−½dθ₆

meaning that the fingers F1 and F2 fold half as fast on itself granting it the ability to open twice as wide with regards to opening angles, whereby the sensor 105 located where shown in FIGS. 11 and 12 may sense forces on the full range of the gripper 100.

Therefore, the robotic gripper 100 has two fingers, such as F1 and/or F2, with two or more phalanges, such as proximal phalanx 20 and distal phalanx 30. The phalanges are articulated using epicycloidal joints between them and with a base. A transmission linkage may help maintaining the distal phalanges 30 parallel when no contact occurs with the environment, if desired. The transmission linkage may include links forming interconnected four-bar parallelograms. In an embodiment, an underactuation mechanism may allow underactuation in one or more of the fingers, of the F2 type. The underactuation is a relative motion of the phalanges with locked input based on linkages, when contact is made with an object. The moniker “underactuation” is used due to the fact that no additional DOA may be required, yet the finger F2 can react in accordance with the grasping sequences of FIG. 4 and of FIG. 5, on the same DOA. The mechanism that allows the underactuation of the finger F2 constructed with epicycloidal joints may be described as a four-bar linkage located at the base 10 of the finger F2, and which provides the required extra freedom while not interfering with the transmission linkage that maintains the phalanges 30 parallel, or in constant orientation, when no contact occurs with the environment. When no contact is present on the inner proximal phalanx 20, the four-bar linkage remains in a constant configuration, i.e., it does not deform as its corner angles remain constant, and this may be used to achieve a parallel grasp between a pair of fingers F2 (or a combination of F1 and F2) if desired, as observed in the grasping sequence of FIG. 4. By contrast, an enveloping grasp is obtained when a contact is present on the proximal phalanx 20 causing the input movement to be transmitted directly to the distal phalanx 30, making it close inward towards the object, as observed in the grasping sequence of FIG. 5.

The fingers F1 and F2 may optionally include the nail system 70, and thus have a bidirectionally driven nail(s) 71 whose motion is coupled to the relative motion between the distal phalanx 30 and a reference on the base 10, which is transmitted through the transmission linkage that maintains the distal phalanges 30 in a constant orientation. This yields an improved scooping of flat objects, for example, because of the nail system 70 causing a deployment of the nail(s) 71 when the distal phalanx 30 is at a desired scooping angle, the nail(s) 71 being deployed throughout the scooping motion. When placing fingers face to face as in FIG. 10, the fingers F1 and F2 can produce a ‘shearing’ motion that allows the grasping of flat objects using a ‘butting’ grasp, with finger F1 serving as abutment, while finger F2 deploys its nail(s) 71.

As observed from exemplary embodiments, the fingers F1 and F2, may be constructed so as to have the gears of the epicycloidal joints located inside the fingers F1 and/or F2 to ensure safety. One of the fingers F1 or F2 in a gripper, such as the gripper 100, may be fitted with a sensor 105, such as a load cell embedded in the links, in which the contact force with the environment is amplified, thereby alleviating the need for force/torque sensors at the base of the finger/gripper. An alternative embodiment, among others, consists in using an encoder on the motor and a second encoder on the finger base joint to infer the applied loads.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure, and such modifications are intended to fall within the appended claims.

1.-43. (canceled)

44. A nail system for a distal phalanx of a mechanical finger, comprising at least one nail having a joint mechanism movably connecting the nail to the distal phalanx between a stowed position in which a grasping tip of the nail is concealed in the distal phalanx, and a deployed configuration in which the grasping tip projects out of the distal phalanx, and a biasing member for returning the nail to the stowed configuration, wherein the nail is moved to the deployed configuration by rotation of the distal phalanx relative to a remainder of the mechanical finger as induced by contact of an object by the distal phalanx.

45. The nail system according to claim 44, wherein the distal phalanx includes a pair of plates, with the nail slidingly positioned between plates.

46. The nail system according to claim 44, wherein the distal phalanx is connected to a gear configured to form an epicyclic joint with a phalanx adjacent to the distal phalanx.

47. The nail system according to claim 46, wherein the distal phalanx is rotatably connected to the gear, and is biased to a given orientation relative to the gear by the biasing member.

48. The nail system according to claim 47, wherein the nail system includes a member received in at least one slot of the nail, the member engaged with the gear such that a rotation of the distal phalanx relative to the gear imparts a movement of the nail to the deployed configuration via a cooperation of the member and the slot.

49. The nail system according to claim 47, including a cam and follower surface assembly between the nail and the gear, the cam being in operative contact with the follower surface, whereby a rotation of the distal phalanx relative to the gear imparts a movement of the nail to the deployed configuration via a cooperation of the cam and follower surface.

50. The nail system according to claim 49, wherein the follower surface is peripheral part of the nail.

51. A mechanical finger comprising:
a base having a base gear,
at least two phalanges, with a first phalanx having a first proximal phalanx gear at a proximal end and a first distal phalanx gear at the distal end, the first proximal phalanx gear operationally coupled to the base gear for the first phalanx to rotate relative to the base gear, and at least a second phalanx having a second proximal phalanx gear at a proximal end, the second proximal phalanx gear operationally coupled to the first distal phalanx gear for the second phalanx to rotate relative to the first phalanx,
a transmission linkage assembly operatively connected to the base and to the at least two phalanges, the transmission linkage assembly including a first carrier between the base gear and the first proximal phalanx gear, and a second carrier between the first distal phalanx gear and the second proximal phalanx gear, and
the nail system according to claim 44 in a distal most one of the at least two phalanges.

52. The mechanical finger according to claim 51, wherein the first carrier is part of a first coupling link between the base and the first phalanx, rotational axes of the first coupling link being coincident with a center of the base gear and with a center of the first proximal phalanx gear.

53. The mechanical finger according to claim 52, wherein the second carrier is defined by a second coupling link between the first phalanx and the second phalanx, rotational axes of the second coupling link being coincident with a center of the first distal phalanx gear and with a center of the second proximal phalanx gear.

54. The mechanical finger according to claim 53, wherein the transmission linkage assembly has at least one four-bar mechanism.

55. The mechanical finger according to claim 54, wherein the four-bar mechanism includes the first coupling link, the second coupling link, the first phalanx, and a transmission link.

56. The mechanical finger according to claim 55, wherein the first coupling link has rotational joints with the base, the first phalanx and the transmission link, the rotational joints being in a triangular arrangement.

57. The mechanical finger according to claim 55, wherein the second coupling link has rotational joints with the first phalanx, the second phalanx and the transmission link, the rotational joints being in a triangular arrangement.

58. A mechanical finger comprising:
a base,
at least a proximal phalanx and a distal phalanx with a first epicyclic joint between the base and proximal phalanx, and a second epicyclic joint between the proximal phalanx and the distal phalanx;
a distal four-bar mechanism including the proximal phalanx, the distal four-bar mechanism coupled to the distal phalanx; and
the nail system according to claim 44 in a distal most one of the phalanges.

59. The mechanical finger according to claim 58, wherein the first epicyclic joint includes a first coupling link between the base and the proximal phalanx.

60. The mechanical finger according to claim 59, wherein the second epicyclic joint includes a second coupling link between the proximal phalanx and the distal phalanx.

61. The mechanical finger according to claim 60, wherein the distal four-bar mechanism includes the first coupling link, the second coupling link, the proximal phalanx, and a transmission link.

62. The mechanical finger according to claim 61, wherein the first coupling link has rotational joints with the base, the proximal phalanx and the transmission link, the rotational joints being in a triangular arrangement.

63. The mechanical finger according to claim 61, wherein the second coupling link has rotational joints with the proximal phalanx, the distal phalanx and the transmission link, the rotational joints being in a triangular arrangement.
